(19) United States
(12) Patent Application Publication
(10) Pub. No.: US 2016/0292270 A1
(43) Pub. Date: Oct. 6, 2016

(54) TRACKING HEART RATE FOR MUSIC SELECTION

(71) Applicant: INTEL CORPORATION, Santa Clara, CA (US)

(72) Inventors: Indira Negi, Sunnyvale, CA (US); Xiaochao Yang, San Jose, CA (US)

(21) Appl. No.: 14/368,715

(22) PCT Filed: Dec. 27, 2013
(86) PCT No.: PCT/US2013/078009

§ 371 Date: Jun. 25, 2014

(51) Int. Cl. G06F 17/30; A61B 5/00; A61B 5/024; G06F 3/01; G06F 3/16

(52) U.S. Cl. CPC G06F 17/30764; G06F 3/011; G06F 3/165; A61B 5/02438; A61B 5/6817; G06F 17/30743; G10H 2240/131; G10H 2210/076

(57) ABSTRACT

Various systems and methods for tracking heart rate for music selection are described herein. A system to track heart rate for music selection comprises a target heart rate module to identify a target heart rate of a user for an activity session; a heart rate monitor module to receive a current heart rate of the user from a sensor coupled to the system; and an audio selection module to determine when a difference between the current heart rate and the target heart rate is larger than a threshold value, and when the difference is larger than the threshold value: identify a beats per minute of a current audio selection; determine a beats per minute to adjust the heart rate of the user from the current heart rate toward the target heart rate; and select a next audio selection from an audio library with a beats per minute similar to the beats per minute to adjust the heart rate.

### TECHNICAL FIELD

Embodiments described herein generally relate to mobile fitness applications and in particular, to tracking heart rate for music selection.

### BACKGROUND

Many people enjoy listening to music while working out. People tend to work out more effectively and have more enjoyment during a workout when listening to music. When working out, a person may naturally move in rhythm with the time of the music. As an example, runners may pace their steps in a cadence that corresponds to the beats of the music. As a result, people who want to run faster may listen to music with faster tempo.

### BRIEF DESCRIPTION OF THE DRAWINGS

In the drawings, which are not necessarily drawn to scale, like numerals may describe similar components in different views. Like numerals having different letter suffixes may represent different instances of similar components. Some embodiments are illustrated by way of example, and not limitation, in the figures of the accompanying drawings in which:

FIG. 1 is a schematic drawing illustrating a system to track heart rate for music selection, according to an embodiment;

FIG. 2 is a block diagram of a PID controller, according to an embodiment;

FIG. 3 is a chart illustrating a process reaction curve showing the calibration of the PID coefficients Kp, Ki, and Kd, according to an embodiment;

FIG. 4 is an example of a user interface to select and customize parameters of a workout, according to an embodiment;

FIG. 5 is an example of a user interface presented to a user during a workout, according to an embodiment;

FIG. 6 is a flowchart illustrating a method for tracking heart rate for music selection, according to an embodiment; and

FIG. 7 is a block diagram illustrating an example machine upon which any one or more of the techniques (e.g., methodologies) discussed herein may perform, according to an example embodiment.

### DETAILED DESCRIPTION

While training or working out, some people like to stay in certain heart rate zones, such as a cardio or fat burning zone. Various heart rate zones are understood to work different biological systems and are used to achieve different physiological results (e.g., weight loss or strength training). Heart rate zones are typically based on a maximum heart rate. The maximum heart rate is the highest heart rate that an individual may achieve before encountering severe problems. The maximum heart rate may be determined by a heart rate stress test or by using a formula to estimate the maximum heart rate. The formula may be based on age, weight, physical condition, type of activity, and other factors. One commonly accepted formula to determine the maximum heart rate is HRₘₐₓ=220−Age. As such, the maximum heart rate for a 35 year old person is approximately 185 beats per minute (BPM).

When a person is working out, the person may experience temporary fatigue or a burst of energy, which may decrease or increase their cadence. Consequently, the person's heart rate may decrease or increase outside of a target heart rate zone. In such situations, music may be used to either energize or pacify the person in order to bring the person's heart rate back into the target heart rate zone. In an embodiment, the music is changed relatively quickly, such as in the middle of a song, to more quickly move the person's heart rate back toward the target heart rate zone.

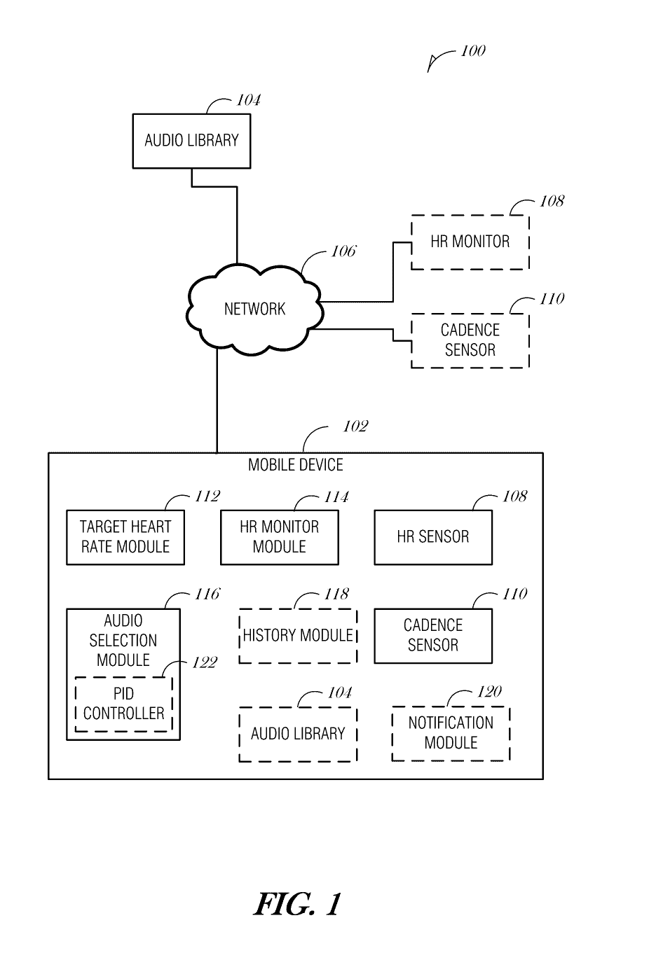

FIG. 1 is a schematic drawing illustrating a system 100 to track heart rate for music selection, according to an embodiment. FIG. 1 includes a mobile device 102 and an audio library 104, communicatively coupled via a network 106.

The mobile device 102 may be a device such as a smartphone, cellular telephone, mobile phone, laptop computer, tablet computer, music player, or other portable networked device. In general, the mobile device 102 is small and light enough to be considered portable and includes a mechanism to connect to a network, either over a persistent or intermittent connection.

The audio library 104 may be remote from the mobile device 102 or stored at the mobile device 102. The audio library 104 may be a collection of one or more audio works, such as music files, stored at the mobile device 102. The audio library 104 may be an online resource, such as a streaming music service or a network storage device.

The network 106 may include local-area networks (LAN), wide-area networks (WAN), wireless networks (e.g., 802.11 or cellular network), the Public Switched Telephone Network (PSTN) network, ad hoc networks, personal area networks (e.g., Bluetooth) or other combinations or permutations of network protocols and network types. The network 108 may include a single local area network (LAN) or wide-area network (WAN), or combinations of LANs or WANs, such as the Internet. The various devices (e.g., mobile device 106 or audio library 104) coupled to the network 108 may be coupled to the network 108 via one or more wired or wireless connections.

The mobile device 102 may include a heart rate monitor 108 and a cadence sensor 110. The heart rate monitor 108 may be incorporated into the mobile device 102 or communicatively coupled to the mobile device 102 with a wired or wireless connection (e.g., via the network 106). The heart rate monitor 108 may be an optical sensor, such as a camera on the mobile device 102 or an optical pulse monitoring sensor in an earphone. The heart rate monitor 108 may also be a chest strap, wrist band, finger band, or other sensor to detect the user's heart rate. The cadence sensor 110 may also be incorporated into the mobile device 102 (e.g., using an accelerometer) or separate from the mobile device 102, such as with a pedometer worn at the waist, and communicatively connected to the mobile device 102.

The mobile device may also include a target heart rate module 112, a heart rate monitor module 114, and an audio selection module 116. In some embodiments, the mobile device 102 may include a history module 118 or a notification module 120.

Various modules 112, 114, 116, 118, 120 may be incorporated or integrated into an application that executes on the mobile device 102. The application may execute in the background and collect data from the sensors and populate a database, which may be accessed by one or more other applications, to enable access to heart rate data. Multiple applications may be developed to use the real-time or historical heart rate data for various purposes.

In operation, the application may query the user or calculate for the user a target heart rate. Then the application may select music from a library based on the beats per minute (BPM) of the music, where the BPM of the music correlates to the target heart rate. If the person exercising does not maintain a heart rate at or near the target heart rate, or in a target heart rate zone, then the application may notify the user. The notification may be an audible notification, such as a voice feedback or a series or one or more beeps or tones. The notification may be presented in a manner that interrupts playback of the current music selection. If the user does not adjust to move their heart rate back into the target heart rate zone or the near the target heart rate, then the application may select a new song based on the then current and target heart rates.

Thus, the application is able to change songs in anticipation of the target heart rate change in order to get the user's heart rate to start moving toward the target sooner. The song selection may be made in part based on the kind of activity that the user is engaged in. So, song selection may be different when the user is running, power walking, or on an exercise bike, for example.

Generally, songs are picked to motivate the user to move with a specific cadence (steps/cycles per minute). Users tend to match their cadence to the BPM of the music they are listening to. This allows very fine control of the cadence. This cadence may be determined either from accelerometer-based sensors in shoes, from the earphones, or sensors embedded in the mobile device 102. It may also be used to coach users to run to a target cadence.

The mobile device 102 is a system to calculate vehicle ratings via measured driver behavior. The target heart rate module 112 may be used to identify a target heart rate of a user for an activity session. In an embodiment, to identify the target heart rate, the target heart rate module 112 is to receive the target heart from the user. For example, the target heart rate module 112 may present a user interface prompt on the mobile device 102 and receive a selection for the target heart rate from the user.

In an embodiment, to identify the target heart rate, the target heart rate module is to calculate the target heart rate based on at least one of an age, a height, or a weight of the user. For example, the target heart rate for aerobic exercise may be calculated as 75% of 220 minus the user's age.

The heart rate monitor module 114 may be used to receive a current heart rate of the user from a sensor coupled to the system. In an embodiment, to receive the current heart rate of the user from the sensor, the heart rate monitor module 114 receives the current heart rate from a heart rate monitor in an earphone. In another embodiment, to receive the current heart rate of the user from the sensor, the heart rate monitor module 114 receives the current heart rate from a heart rate monitor chest strap.

The audio selection module 116 may be used to determine when a difference between the current heart rate and the target heart rate is larger than a threshold value, and when the difference is larger than the threshold value for a duration longer than a threshold time, identify a beats per minute of a current audio selection, determine a beats per minute to adjust the heart rate of the user from the current heart rate toward the target heart rate, and select a next audio selection from an audio library with a beats per minute similar to the beats per minute to adjust the heart rate. In an embodiment, the threshold value is ten heart beats per minute. In an embodiment, the threshold value is user adjustable. As such, if the target heart rate is 180, then the threshold values are +/−18 BPM from 180. It is understood that other thresholds may be used and that the threshold may be expressed in terms of heart rate BPM or percentages of a max BPM, for example.

In an embodiment, to identify the beats per minute of the current audio selection, the audio selection module 116 is to access metadata of the current audio selection. Metadata may be stored in an electronic file with the audio track of an audio selection.

In an embodiment, to identify the beats per minute of the current audio selection, the audio selection module 116 is to perform acoustic analysis of the audio selection. For example, the audio selection module 116 may play a portion of the current audio selection and analyze the portion to determine a BPM of the portion.

In an embodiment, to determine the beats per minute to adjust the heart rate of the user from the current heart rate toward the target heart rate, the audio selection module 116 is to use a feedback controller to calculate the beats per minute. In a further embodiment, the feedback controller comprises a proportional integral derivative (PID) controller 122. In an embodiment, the PID controller 122 includes a plurality of coefficients. In an embodiment, the plurality of coefficients is calculated during a calibration activity performed by the user. For example, the user may be requested to perform a calibration exercise session so that the coefficients are tuned to the user. In an embodiment, the plurality of coefficients comprise Kp, Ki, and Kd, where Kp represents a present error between the current heart rate and the target heart rate, Ki represents an accumulation of past errors between the current heart rate and the target heart rate, and Kp represents a prediction of future errors between the current heart rate and the target heart rate.

In an embodiment, to select the next audio selection from the audio library, the audio selection module 116 is to identify a potential next audio selection from the audio library. The potential next audio selection may be selected based on a playlist the user is listening to from the audio library. The audio selection module 116 may then analyze the potential next audio selection to determine whether the beats per minute substantially matches the beats per minute to adjust the heart rate when the beats per minute of the potential next audio selection substantially matches the beats per minute to adjust the heart rate, select the potential next audio selection from the audio library as the next audio selection. To substantially match the BPM of the next audio selection, the potential next audio selection may be required to be within some small threshold value of the BPM of the next audio selection, such as three BPM. In an embodiment, to analyze the potential next audio selection, the audio selection module 116 is to perform acoustic analysis of the potential next audio selection. This allows the user to access an audio library that has music with various BPM values without the need to pre-filter the music. This also allows the mobile device 102 to be more flexible and dynamically use music that is appropriate for the situation.

The next music selection may be made while the user is listening to a current music selection so as to avoid interruptions in the music playback, and consequently, interrupts to the user's workout. Thus, in an embodiment, the next audio selection is selected during playback of the current audio selection.

In an embodiment, the target heart rate changes at a particular interval and the audio selection module 116 identifies the beats per minute of the current audio selection, determines the beats per minute, and selects the next audio selection at the particular interval. The particular interval may be set by the user, such as for use in interval training. The interval may be any amount of time, such as 5 minutes.

In an embodiment, the mobile device includes a history module 118 to store a history of audio selections played to the user, and when the potential next selection is in the history of audio selections, the audio selection module 116 may select a different potential next selection to analyze beats per minute. This avoids playing the same audio to the user, which may annoy, distract, or bore the user.

In an embodiment, the mobile device 102 includes a notification module 120 to present a notification to the user when the difference between the current heart rate and the target heart rate is larger than the threshold value. The notification may be an audible one, such as with a voice or an alarm, tactile (e.g., vibrate a smartphone), haptic, or visual (e.g., a warning dialog box), or any combination. The notification may include information about the user's performance, such as heart rate over time or current heart rate, or encouragement to assist the user to maintain or regain the target heart rate. When a timeout period expires after the notification is presented to the user, the audio selection module 116 may evaluate the difference between the current heart rate and the target heart rate to determine whether it has been larger than the threshold value for the timeout period, and is so, perform the identifying the beats per minute of the current audio selection, determining the beats per minute to adjust the heart rate of the user from the current heart rate toward the target heart rate, and selecting the next audio selection from the audio library. In an embodiment, the timeout period is 40 seconds. The timeout period may be user adjustable. The timeout period is used to ensure that the user has a chance to respond to the notification before changing music. Thus, if a user does not respond to the notification, new music may be selected to encourage the user to increase or decrease their cadence, in order to bring their heart rate toward the target.

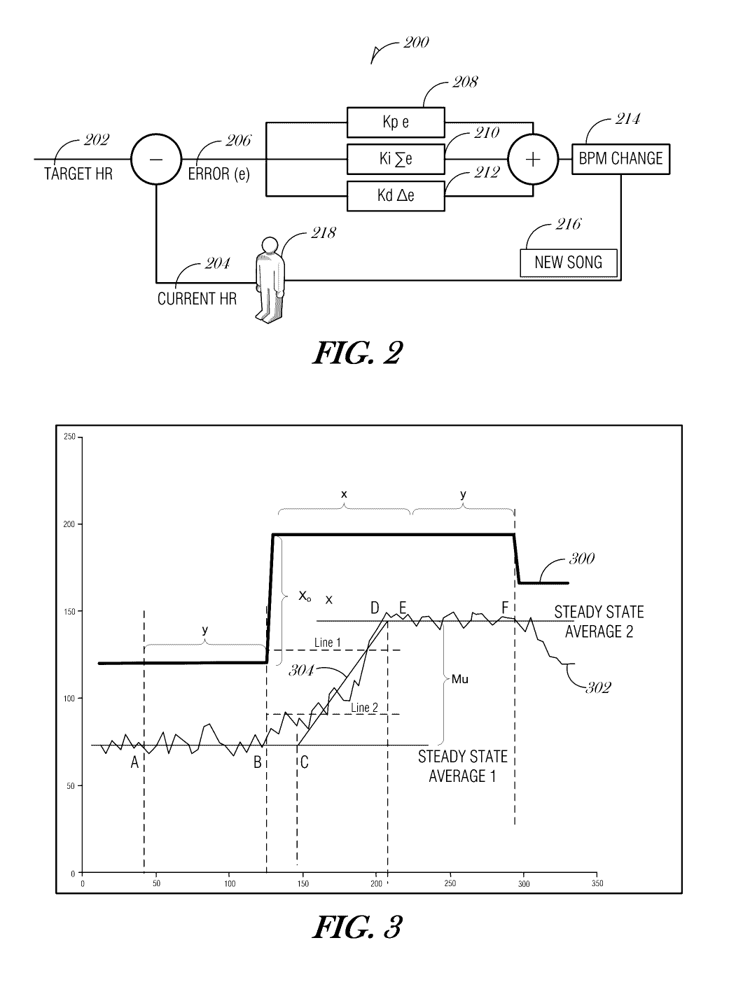

FIG. 2 is a block diagram of a PID controller 200, according to an embodiment. The PID controller 200 includes an input target heart rate 202 and a current heart rate 204, the difference resulting in an error value (e) 206, which is used with the coefficients Kp 208, Ki 210, and Kd 212. The coefficients 208, 210, 212 are aggregated and a BPM change 214 is calculated. A new song 216 is selected based on the BPM change and presented to the user 218. The aim of the music selection is to minimize the error 206.

The PID controller coefficients may be calibrated. In an embodiment, the Ziegler-Nichols method is employed to tune the PID coefficients based on the process reaction curve for each target transition. The PID coefficients are calculated for a user based on how reactive a user's heart rate is to a change in song BPM. The calibration process is explained with the help of FIG. 3.

FIG. 3 is a chart illustrating a process reaction curve showing the calibration of the PID coefficients Kp, Ki, and Kd, according to an embodiment. The x-axis and y-axis of the chart are in seconds. Curve 300 shows the song BPM. This is the control input to the PID controller. When the song BPM changes by Xₒ, the user's HR 302 starts to rise in response. To calculate the steady state value for an interval, y data points are averaged, starting x data points after the disturbance. In FIG. 3, x=85 seconds, so 85 seconds after a song changes (e.g., the disturbance), the user's HR is assumed to have reached steady state. Steady State Average 1 is the average of points from A to B and Steady State Average 2 is the average of points from D to F. Mu is the difference between the new and previous steady states for the user's HR 302. Line 1 is Steady State Average 1+25% Mu, and line 2 is Steady State Average 2−25% Mu. The line tangent 304 to the response curve is approximated by the intersection of the smoothed response curve with line1 and line2. Where this tangent line 304 intersects with the two steady states are points C and D.

In FIG. 3, τ_{dead} is the time from B to C, e.g., the time taken from the moment the disturbance was introduced to the first sign of change, t is the time from C to D, e.g., the time for the response to occur, and Kp=1.2 (Xₒ τ)/(Mu τ_{dead}), Ki=Kp/(2 τ_{dead}) and Kd=0.5 Kp τ_{dead}.

The calibration may be done during a calibration run and the coefficients may be tuned during a workout. This ensures the constants stay realistic throughout a long workout, where varying degrees of fatigue will change how the user's HR reacts to music.

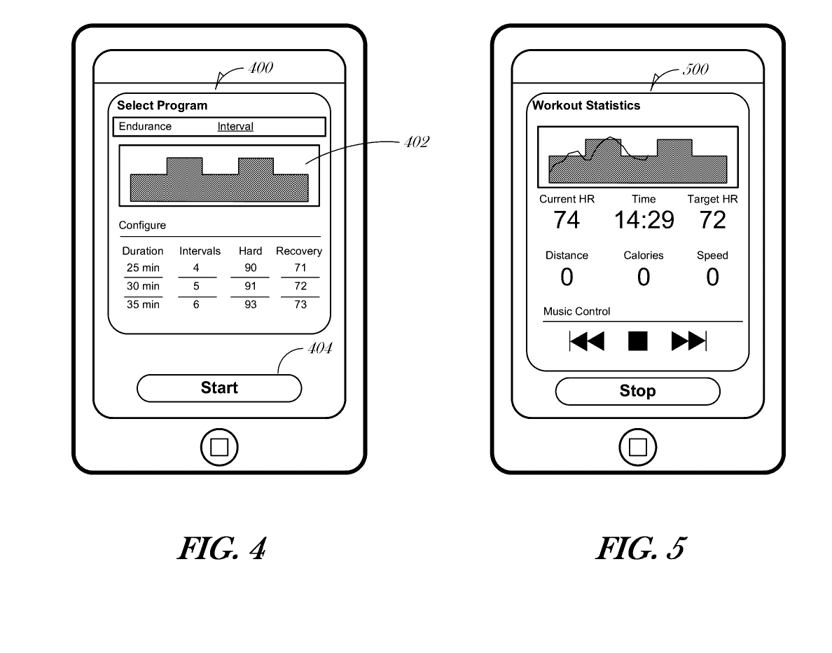

FIGS. 4 and 5 illustrate user interfaces, according to an example embodiment. FIG. 4 is an example of a user interface 400 to select and customize parameters of a workout. The user may select between an endurance workout and an interval workout. An endurance workout is one where the user maintains a relatively static heart rate over a long period of time (e.g., two hours). An interval workout is one where the user works out at a high heart rate for a period of time and then interlaces the hard workouts with recovery periods, where the user's heart rate is lowered for a period. The recovery period may be the same length as the hard workout period, or may be a longer or shorter length. The user interface 400 shown in FIG. 4 is to configure an interval workout. The user interface 400 includes a visual indication of the selections 402 to assist the user when configuring the workout. The user may select the duration of the workout, the number of intervals in the duration, the target heart rate during the hard intervals, and the target heart rate during the recovery intervals. After selecting the parameters, the user may activate the “Start” control 404 to begin the workout.

FIG. 5 is an example of a user interface 500 presented to a user during a workout, according to an embodiment. The user interface 500 displays the current heart rate, the time elapsed in the workout, the target heart rate, the distance travelled, the estimated calories burned, and the current speed of the user. In the example shown in FIG. 5, the user is performing a relatively stationary exercise, such running on a treadmill, so the distance, calories, and speed are all zero (calories may be estimated based on speed and distance travelled in a given time). The user interface 500 also includes music controls, which may include the name of the artist or the name of the song, in addition to other information, such as beats per minute of the song, in various embodiments. In an embodiment, the user interface 500 is presented on a lock screen of the mobile device 102, so that the user does not have to unlock the mobile device 102 in order to view the user interface 500.

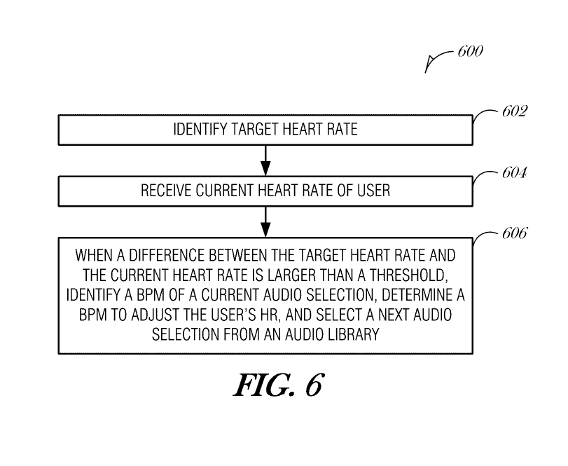

FIG. 6 is a flowchart illustrating a method 600 method for tracking heart rate for music selection, according to an embodiment. At 602, a target heart rate of a user for an activity session is identified at a mobile device. The activity session may be an endurance or interval training session, such as described above in FIGS. 4-5. In an embodiment, identifying the target heart rate comprises receiving the target heart from the user. In an embodiment, identifying the target heart rate comprises calculating the target heart rate based on at least one of an age, a height, or a weight of the user.

At 604, a current heart rate of the user is received from a sensor coupled to the mobile device. In an embodiment, receiving the current heart rate of the user from the sensor comprises receiving the current heart rate from a heart rate monitor in an earphone. In an embodiment, receiving the current heart rate of the user from the sensor comprises receiving the current heart rate from a heart rate monitor chest strap.

At 606, when a difference between the current heart rate and the target heart rate is larger than a threshold value for a duration longer than a threshold time, a beats per minute of a current audio selection is identified. In an embodiment, the threshold value is ten heart beats per minute. In an embodiment, the threshold value is user adjustable. In an embodiment, identifying the beats per minute of the current audio selection comprises accessing metadata of the current audio selection. In an embodiment, identifying the beats per minute of the current audio selection comprises performing acoustic analysis of the audio selection.

Then, a beats per minute to adjust the heart rate of the user from the current heart rate toward the target heart rate is determined. In an embodiment, determining the beats per minute to adjust the heart rate of the user from the current heart rate toward the target heart rate comprises using a feedback controller to calculate the beats per minute. In an embodiment, feedback controller comprises a proportional integral derivative (PID) controller. In an embodiment, the PID controller includes a plurality of coefficients. In an embodiment, the plurality of coefficients is calculated during a calibration activity performed by the user. In an embodiment, the plurality of coefficients comprise Kp, Ki, and Kd, where Kp represents a present error between the current heart rate and the target heart rate, Ki represents an accumulation of past errors between the current heart rate and the target heart rate, and Kp represents a prediction of future errors between the current heart rate and the target heart rate.

Then, a next audio selection is selected from an audio library with a beats per minute similar to the beats per minute to adjust the heart rate. In an embodiment, selecting the next audio selection from the audio library comprises identifying a potential next audio selection from the audio library, analyzing the potential next audio selection to determine whether the beats per minute of the potential next audio selection substantially matches the beats per minute to adjust the heart rate, and when the beats per minute of the potential next audio selection substantially matches the beats per minute to adjust the heart rate, selecting the potential next audio selection from the audio library as the next audio selection. In an embodiment, analyzing the potential next audio selection comprises performing acoustic analysis of the potential next audio selection.

In an embodiment, selecting the next audio selection is performed during playback of the current audio selection.

In an embodiment, the target heart rate changes at a particular interval and the identifying the beats per minute of the current audio selection, determining the beats per minute, and selecting the next audio selection are performed at the particular interval.

In an embodiment, the method 600 includes storing a history of audio selections played to the user and when the potential next selection is in the history of audio selections, selecting a different potential next selection to analyze beats per minute.

In an embodiment, the method 600 includes presenting a notification to the user when the difference between the current heart rate and the target heart rate is larger than the threshold value. In a further embodiment, when a timeout period expires after the notification is presented to the user, evaluating the difference between the current heart rate and the target heart rate to determine whether it has been larger than the threshold value for the timeout period, and is so, performing the identifying the beats per minute of the current audio selection, determining the beats per minute to adjust the heart rate of the user from the current heart rate toward the target heart rate, and selecting the next audio selection from the audio library.

Embodiments may be implemented in one or a combination of hardware, firmware, and software. Embodiments may also be implemented as instructions stored on a machine-readable storage device, which may be read and executed by at least one processor to perform the operations described herein. A machine-readable storage device may include any non-transitory mechanism for storing information in a form readable by a machine (e.g., a computer). For example, a machine-readable storage device may include read-only memory (ROM), random-access memory (RAM), magnetic disk storage media, optical storage media, flash-memory devices, and other storage devices and media.

Examples, as described herein, may include, or may operate on, logic or a number of components, modules, or mechanisms. Modules may be hardware, software, or firmware communicatively coupled to one or more processors in order to carry out the operations described herein. Modules may hardware modules, and as such modules may be considered tangible entities capable of performing specified operations and may be configured or arranged in a certain manner. In an example, circuits may be arranged (e.g., internally or with respect to external entities such as other circuits) in a specified manner as a module. In an example, the whole or part of one or more computer systems (e.g., a standalone, client or server computer system) or one or more hardware processors may be configured by firmware or software (e.g., instructions, an application portion, or an application) as a module that operates to perform specified operations. In an example, the software may reside on a machine-readable medium. In an example, the software, when executed by the underlying hardware of the module, causes the hardware to perform the specified operations. Accordingly, the term hardware module is understood to encompass a tangible entity, be that an entity that is physically constructed, specifically configured (e.g., hardwired), or temporarily (e.g., transitorily) configured (e.g., programmed) to operate in a specified manner or to perform part or all of any operation described herein. Considering examples in which modules are temporarily configured, each of the modules need not be instantiated at any one moment in time. For example, where the modules comprise a general-purpose hardware processor configured using software; the general-purpose hardware processor may be configured as respective different modules at different times. Software may accordingly configure a hardware processor, for example, to constitute a particular module at one instance of time and to constitute a different module at a different instance of time. Modules may also be software or firmware modules, which operate to perform the methodologies described herein.

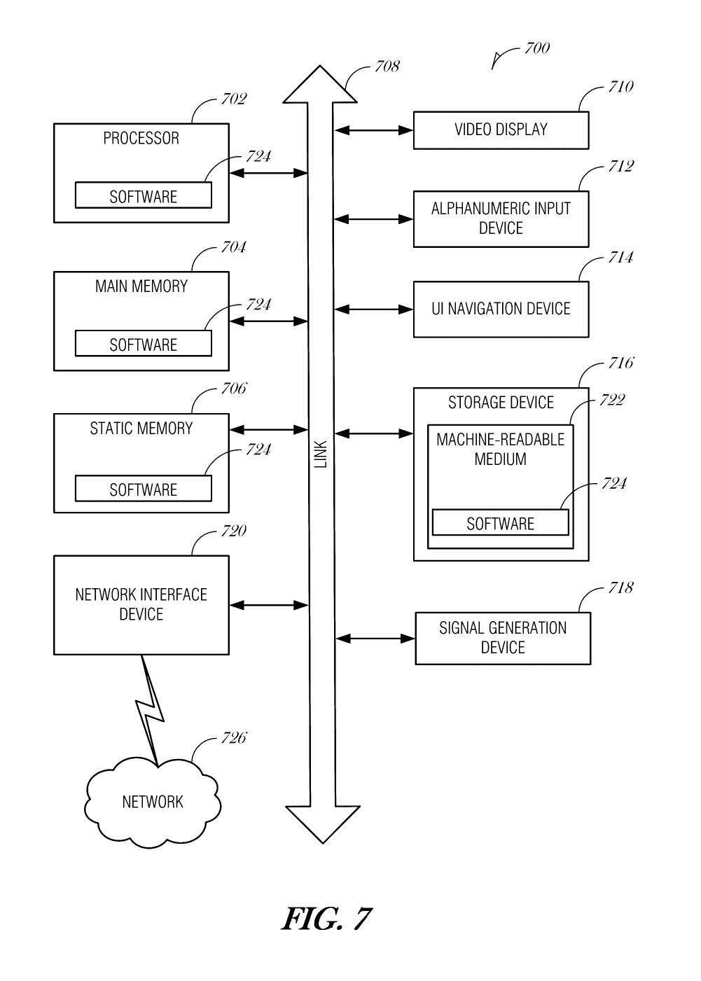

FIG. 7 is a block diagram illustrating a machine in the example form of a computer system 700, within which a set or sequence of instructions may be executed to cause the machine to perform any one of the methodologies discussed herein, according to an example embodiment. In alternative embodiments, the machine operates as a standalone device or may be connected (e.g., networked) to other machines. In a networked deployment, the machine may operate in the capacity of either a server or a client machine in server-client network environments, or it may act as a peer machine in peer-to-peer (or distributed) network environments. The machine may be an onboard vehicle system, wearable device, personal computer (PC), a tablet PC, a hybrid tablet, a personal digital assistant (PDA), a mobile telephone, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. Similarly, the term “processor-based system” shall be taken to include any set of one or more machines that are controlled by or operated by a processor (e.g., a computer) to individually or jointly execute instructions to perform any one or more of the methodologies discussed herein.

Example computer system 700 includes at least one processor 702 (e.g., a central processing unit (CPU), a graphics processing unit (GPU) or both, processor cores, compute nodes, etc.), a main memory 704 and a static memory 706, which communicate with each other via a link 708 (e.g., bus). The computer system 700 may further include a video display unit 710, an alphanumeric input device 712 (e.g., a keyboard), and a user interface (UI) navigation device 714 (e.g., a mouse). In one embodiment, the video display unit 710, input device 712 and UI navigation device 714 are incorporated into a touch screen display. The computer system 700 may additionally include a storage device 716 (e.g., a drive unit), a signal generation device 718 (e.g., a speaker), a network interface device 720, and one or more sensors (not shown), such as a global positioning system (GPS) sensor, compass, accelerometer, or other sensor.

The storage device 716 includes a machine-readable medium 722 on which is stored one or more sets of data structures and instructions 724 (e.g., software) embodying or utilized by any one or more of the methodologies or functions described herein. The instructions 724 may also reside, completely or at least partially, within the main memory 704, static memory 706, and/or within the processor 702 during execution thereof by the computer system 700, with the main memory 704, static memory 706, and the processor 702 also constituting machine-readable media.

While the machine-readable medium 722 is illustrated in an example embodiment to be a single medium, the term “machine-readable medium” may include a single medium or multiple media (e.g., a centralized or distributed database, and/or associated caches and servers) that store the one or more instructions 724. The term “machine-readable medium” shall also be taken to include any tangible medium that is capable of storing, encoding or carrying instructions for execution by the machine and that cause the machine to perform any one or more of the methodologies of the present disclosure or that is capable of storing, encoding or carrying data structures utilized by or associated with such instructions. The term “machine-readable medium” shall accordingly be taken to include, but not be limited to, solid-state memories, and optical and magnetic media. Specific examples of machine-readable media include non-volatile memory, including but not limited to, by way of example, semiconductor memory devices (e.g., electrically programmable read-only memory (EPROM), electrically erasable programmable read-only memory (EEPROM)) and flash memory devices; magnetic disks such as internal hard disks and removable disks; magneto-optical disks; and CD-ROM and DVD-ROM disks.

The instructions 724 may further be transmitted or received over a communications network 726 using a transmission medium via the network interface device 720 utilizing any one of a number of well-known transfer protocols (e.g., HTTP). Examples of communication networks include a local area network (LAN), a wide area network (WAN), the Internet, mobile telephone networks, plain old telephone (POTS) networks, and wireless data networks (e.g., Wi-Fi, 3G, and 4G LTE/LTE-A or WiMAX networks). The term “transmission medium” shall be taken to include any intangible medium that is capable of storing, encoding, or carrying instructions for execution by the machine, and includes digital or analog communications signals or other intangible medium to facilitate communication of such software.

### Additional Notes & Examples

Example 1 includes subject matter (such as a device, apparatus, or machine) comprising a system to track heart rate for music selection, comprising a target heart rate module to identify a target heart rate of a user for an activity session; a heart rate monitor module to receive a current heart rate of the user from a sensor coupled to the system; and an audio selection module to determine when a difference between the current heart rate and the target heart rate is larger than a threshold value, and when the difference is larger than the threshold value for a duration longer than a threshold time: identify a beats per minute of a current audio selection; determine a beats per minute to adjust the heart rate of the user from the current heart rate toward the target heart rate; and select a next audio selection from an audio library with a beats per minute similar to the beats per minute to adjust the heart rate.

In Example 2, the subject matter of Example 1 may optionally include, wherein to identify the target heart rate, the target heart rate module is to receive the target heart from the user.

In Example 3, the subject matter of any one or more of Examples 1 to 2 may optionally include, wherein to identify the target heart rate, the target heart rate module is to calculate the target heart rate based on at least one of an age, a height, or a weight of the user.

In Example 4, the subject matter of any one or more of Examples 1 to 3 may optionally include, wherein the target heart rate changes at a particular interval and wherein the audio selection module identifies the beats per minute of the current audio selection, determines the beats per minute of the next audio selection, and selects the next audio selection at the particular interval.

In Example 5, the subject matter of any one or more of Examples 1 to 4 may optionally include, wherein to receive the current heart rate of the user from the sensor, the heart rate monitor module receives the current heart rate from a heart rate monitor in an earphone.

In Example 6, the subject matter of any one or more of Examples 1 to 5 may optionally include, wherein to receive the current heart rate of the user from the sensor, the heart rate monitor module receives the current heart rate from a heart rate monitor chest strap.

In Example 7, the subject matter of any one or more of Examples 1 to 6 may optionally include, wherein the threshold value is ten heart beats per minute.

In Example 8, the subject matter of any one or more of Examples 1 to 7 may optionally include, wherein to identify the beats per minute of the current audio selection, the audio selection module is to access metadata of the current audio selection.

In Example 9, the subject matter of any one or more of Examples 1 to 8 may optionally include, wherein to identify the beats per minute of the current audio selection, the audio selection module is to perform acoustic analysis of the audio selection.

In Example 10, the subject matter of any one or more of Examples 1 to 9 may optionally include, wherein to determine the beats per minute to adjust the heart rate of the user from the current heart rate toward the target heart rate, the audio selection module is to use a feedback controller to calculate the beats per minute.

In Example 11, the subject matter of any one or more of Examples 1 to 10 may optionally include, wherein the feedback controller comprises a proportional integral derivative (PID) controller.

In Example 12, the subject matter of any one or more of Examples 1 to 11 may optionally include, wherein the PID controller includes a plurality of coefficients.

In Example 13, the subject matter of any one or more of Examples 1 to 12 may optionally include, wherein the plurality of coefficients is calculated during a calibration activity performed by the user.

In Example 14, the subject matter of any one or more of Examples 1 to 13 may optionally include, wherein the plurality of coefficients comprise Kp, Ki, and Kd, where Kp represents a present error between the current heart rate and the target heart rate, Ki represents an accumulation of past errors between the current heart rate and the target heart rate, and Kp represents a prediction of future errors between the current heart rate and the target heart rate.

In Example 15, the subject matter of any one or more of Examples 1 to 14 may optionally include, wherein to select the next audio selection from the audio library, the audio selection module is to: identify a potential next audio selection from the audio library; analyze the potential next audio selection to determine whether the beats per minute of the potential next audio selection substantially matches the beats per minute to adjust the heart rate; and when the beats per minute of the potential next audio selection substantially matches the beats per minute to adjust the heart rate, select the potential next audio selection from the audio library as the next audio selection.

In Example 16, the subject matter of any one or more of Examples 1 to 15 may optionally include, wherein to analyze the potential next audio selection, the audio selection module is to perform acoustic analysis of the potential next audio selection.

In Example 17, the subject matter of any one or more of Examples 1 to 16 may optionally include, wherein the next audio selection is selected during playback of the current audio selection.

In Example 18, the subject matter of any one or more of Examples 1 to 17 may optionally include, a history module to store a history of audio selections played to the user, and when the potential next selection is in the history of audio selections, the audio selection module is to select a different potential next selection to analyze beats per minute.

In Example 19, the subject matter of any one or more of Examples 1 to 18 may optionally include, a notification module to present a notification to the user when the difference between the current heart rate and the target heart rate is larger than the threshold value.

In Example 20, the subject matter of any one or more of Examples 1 to 19 may optionally include, wherein when a timeout period expires after the notification is presented to the user, the audio selection module evaluates the difference between the current heart rate and the target heart rate to determine whether it has been larger than the threshold value for the timeout period, and is so, performs the identifying the beats per minute of the current audio selection, determining the beats per minute of the next audio selection to adjust the heart rate of the user from the current heart rate toward the target heart rate, and selecting the next audio selection from the audio library.

Example 21 includes subject matter for tracking heart rate for music selection (such as a method, means for performing acts, machine readable medium including instructions that when performed by a machine cause the machine to performs acts, or an apparatus configured to perform) comprising identifying, at a mobile device, a target heart rate of a user for an activity session; receiving a current heart rate of the user from a sensor coupled to the mobile device; and when a difference between the current heart rate and the target heart rate is larger than a threshold value for a duration longer than a threshold time: identifying, at the mobile device, a beats per minute of a current audio selection; determining, at the mobile device, a beats per minute to adjust the heart rate of the user from the current heart rate toward the target heart rate; and selecting, at the mobile device, a next audio selection from an audio library with a beats per minute similar to the beats per minute to adjust the heart rate.

In Example 22, the subject matter of Example 21 may optionally include, wherein identifying the target heart rate comprises receiving the target heart from the user.

In Example 23, the subject matter of any one or more of Examples 21 to 22 may optionally include, wherein identifying the target heart rate comprises calculating the target heart rate based on at least one of an age, a height, or a weight of the user.

In Example 24, the subject matter of any one or more of Examples 21 to 23 may optionally include, wherein the target heart rate changes at a particular interval and wherein the identifying the beats per minute of the current audio selection, determining the beats per minute, and selecting the next audio selection are performed at the particular interval.

In Example 25, the subject matter of any one or more of Examples 21 to 24 may optionally include, wherein receiving the current heart rate of the user from the sensor comprises receiving the current heart rate from a heart rate monitor in an earphone.

In Example 26, the subject matter of any one or more of Examples 21 to 25 may optionally include, wherein receiving the current heart rate of the user from the sensor comprises receiving the current heart rate from a heart rate monitor chest strap.

In Example 27, the subject matter of any one or more of Examples 21 to 26 may optionally include, wherein the threshold value is ten heart beats per minute.

In Example 28, the subject matter of any one or more of Examples 21 to 27 may optionally include, wherein identifying the beats per minute of the current audio selection comprises accessing metadata of the current audio selection.

In Example 29, the subject matter of any one or more of Examples 21 to 28 may optionally include, wherein identifying the beats per minute of the current audio selection comprises performing acoustic analysis of the audio selection.

In Example 30, the subject matter of any one or more of Examples 21 to 29 may optionally include, wherein determining the beats per minute to adjust the heart rate of the user from the current heart rate toward the target heart rate comprises using a feedback controller to calculate the beats per minute.

In Example 31, the subject matter of any one or more of Examples 21 to 30 may optionally include, wherein the feedback controller comprises a proportional integral derivative (PID) controller.

In Example 32, the subject matter of any one or more of Examples 21 to 31 may optionally include, wherein the PID controller includes a plurality of coefficients.

In Example 33, the subject matter of any one or more of Examples 21 to 32 may optionally include, wherein the plurality of coefficients is calculated during a calibration activity performed by the user.

In Example 34, the subject matter of any one or more of Examples 21 to 33 may optionally include, wherein the plurality of coefficients comprise Kp, Ki, and Kd, where Kp represents a present error between the current heart rate and the target heart rate, Ki represents an accumulation of past errors between the current heart rate and the target heart rate, and Kp represents a prediction of future errors between the current heart rate and the target heart rate.

In Example 35, the subject matter of any one or more of Examples 21 to 34 may optionally include, wherein selecting the next audio selection from the audio library comprises: identifying a potential next audio selection from the audio library; analyzing the potential next audio selection to determine whether the beats per minute of the potential next audio selection substantially matches the beats per minute to adjust the heart rate; and when the beats per minute of the potential next audio selection substantially matches the beats per minute to adjust the heart rate, selecting the potential next audio selection from the audio library as the next audio selection.

In Example 36, the subject matter of any one or more of Examples 21 to 35 may optionally include, wherein analyzing the potential next audio selection comprises performing acoustic analysis of the potential next audio selection.

In Example 37, the subject matter of any one or more of Examples 21 to 36 may optionally include, wherein selecting the next audio selection is performed during playback of the current audio selection.

In Example 38, the subject matter of any one or more of Examples 21 to 37 may optionally include, storing a history of audio selections played to the user; and when the potential next selection is in the history of audio selections, selecting a different potential next selection to analyze beats per minute.

In Example 39, the subject matter of any one or more of Examples 21 to 38 may optionally include, presenting a notification to the user when the difference between the current heart rate and the target heart rate is larger than the threshold value.

In Example 40, the subject matter of any one or more of Examples 21 to 39 may optionally include, when a timeout period expires after the notification is presented to the user, evaluating the difference between the current heart rate and the target heart rate to determine whether it has been larger than the threshold value for the timeout period, and is so, performing the identifying the beats per minute of the current audio selection, determining the beats per minute to adjust the heart rate of the user from the current heart rate toward the target heart rate, and selecting the next audio selection from the audio library.

Example 41 includes a machine-readable medium including instructions for tracking heart rate for music selection, which when executed by a machine, cause the machine to perform any one of Examples 21-40.

Example 42 includes an apparatus to track heart rate for music selection, the apparatus means for performing any one of Examples 21-40.

Example 43 includes an apparatus to track heart rate for music selection comprising: means for identifying, at a mobile device, a target heart rate of a user for an activity session; means for receiving a current heart rate of the user from a sensor coupled to the mobile device; and means for when a difference between the current heart rate and the target heart rate is larger than a threshold value: means for identifying, at the mobile device, a beats per minute of a current audio selection; means for determining, at the mobile device, a beats per minute to adjust the heart rate of the user from the current heart rate toward the target heart rate; and means for selecting, at the mobile device, a next audio selection from an audio library with a beats per minute similar to the beats per minute to adjust the heart rate.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, also contemplated are examples that include the elements shown or described. Moreover, also contemplated are examples using any combination or permutation of those elements shown or described (or one or more aspects thereof), either with respect to a particular example (or one or more aspects thereof), or with respect to other examples (or one or more aspects thereof) shown or described herein.

Publications, patents, and patent documents referred to in this document are incorporated by reference herein in their entirety, as though individually incorporated by reference. In the event of inconsistent usages between this document and those documents so incorporated by reference, the usage in the incorporated reference(s) are supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to suggest a numerical order for their objects.

The above description is intended to be illustrative, and not restrictive. For example, the above-described examples (or one or more aspects thereof) may be used in combination with others. Other embodiments may be used, such as by one of ordinary skill in the art upon reviewing the above description. The Abstract is to allow the reader to quickly ascertain the nature of the technical disclosure, for example, to comply with 37 C.F.R. §1.72(b) in the United States of America. It is submitted with the understanding that it will not be used to interpret or limit the scope or meaning of the claims. Also, in the above Detailed Description, various features may be grouped together to streamline the disclosure. However, the claims may not set forth every feature disclosed herein as embodiments may feature a subset of said features. Further, embodiments may include fewer features than those disclosed in a particular example. Thus, the following claims are hereby incorporated into the Detailed Description, with a claim standing on its own as a separate embodiment. The scope of the embodiments disclosed herein is to be determined with reference to the appended claims, along with the full scope of equivalents to which such claims are entitled.

1.-25. (canceled)

26. A machine-readable medium including instructions for tracking heart rate for music selection, which when executed by a machine, cause the machine to:
identify, at a mobile device, a target heart rate of a user for an activity session;
receive a current heart rate of the user from a sensor coupled to the mobile device; and
when a difference between the current heart rate and the target heart rate is larger than a threshold value:
identify, at the mobile device, a beats per minute of a current audio selection;
determine, at the mobile device, a beats per minute to adjust the heart rate of the user from the current heart rate toward the target heart rate; and
select, at the mobile device, a next audio selection from an audio library with a beats per minute similar to the beats per minute to adjust the heart rate.

27. The machine-readable medium of claim 26, wherein the target heart rate changes at a particular interval and wherein the instructions to identify the beats per minute of the current audio selection, determine the beats per minute, and select the next audio selection are performed at the particular interval.

28. The machine-readable medium of claim 26, wherein instructions to receive the current heart rate of the user from the sensor comprise instructions to receive the current heart rate from a heart rate monitor in an earphone.

29. The machine-readable medium of claim 26, wherein the threshold value is ten heart beats per minute.

30. The machine-readable medium of claim 26, wherein instructions to identify the beats per minute of the current audio selection comprise instructions to access metadata of the current audio selection.

31. The machine-readable medium of claim 26, wherein instructions to identify the beats per minute of the current audio selection comprise instructions to perform acoustic analysis of the audio selection.

32. The machine-readable medium of claim 26, wherein instructions to determine the beats per minute to adjust the heart rate of the user from the current heart rate toward the target heart rate comprise instructions to use a feedback controller to calculate the beats per minute.

33. The machine-readable medium of claim 32, wherein the feedback controller comprises a proportional integral derivative (PID) controller.

34. The machine-readable medium of claim 33, wherein the PID controller includes a plurality of coefficients.

35. The machine-readable medium of claim 26, wherein instructions to select the next audio selection from the audio library comprise instructions to:
identify a potential next audio selection from the audio library;
analyze the potential next audio selection to determine whether the beats per minute of the potential next audio selection substantially matches the beats per minute to adjust the heart rate; and
when the beats per minute of the potential next audio selection substantially matches the beats per minute to adjust the heart rate, select the potential next audio selection from the audio library as the next audio selection.

36. The machine-readable medium of claim 35, wherein instructions to analyze the potential next audio selection comprise instructions to perform acoustic analysis of the potential next audio selection.

37. The machine-readable medium of claim 35, wherein the instructions to select the next audio selection are performed during playback of the current audio selection.

38. The machine-readable medium of claim 35, further comprising instructions to:
store a history of audio selections played to the user; and
when the potential next selection is in the history of audio selections, select a different potential next selection to analyze beats per minute.

39. The machine-readable medium of claim 26, further comprising instructions to:
present a notification to the user when the difference between the current heart rate and the target heart rate is larger than the threshold value.

40. The machine-readable medium of claim 39, further comprising instructions to:
when a timeout period expires after the notification is presented to the user, evaluate the difference between the current heart rate and the target heart rate to determine whether it has been larger than the threshold value for the timeout period, and is so, performing the identifying the beats per minute of the current audio selection, determining the beats per minute to adjust the heart rate of the user from the current heart rate toward the target heart rate, and selecting the next audio selection from the audio library.

41. A system to track heart rate for music selection, the system comprising:
a target heart rate module to identify a target heart rate of a user for an activity session;
a heart rate monitor module to receive a current heart rate of the user from a sensor coupled to the system; and
an audio selection module to determine when a difference between the current heart rate and the target heart rate is larger than a threshold value, and when the difference is larger than the threshold value:
identify a beats per minute of a current audio selection;
determine a beats per minute to adjust the heart rate of the user from the current heart rate toward the target heart rate; and
select a next audio selection from an audio library with a beats per minute similar to the beats per minute to adjust the heart rate.

42. The system of claim 41, wherein to determine the beats per minute to adjust the heart rate of the user from the current heart rate toward the target heart rate, the audio selection module is to use a feedback controller to calculate the beats per minute.

43. The system of claim 42, wherein the feedback controller comprises a proportional integral derivative (PID) controller.

44. A method for tracking heart rate for music selection, the method comprising:
identifying, at a mobile device, a target heart rate of a user for an activity session;
receiving a current heart rate of the user from a sensor coupled to the mobile device; and
when a difference between the current heart rate and the target heart rate is larger than a threshold value:
identifying, at the mobile device, a beats per minute of a current audio selection;
determining, at the mobile device, a beats per minute to adjust the heart rate of the user from the current heart rate toward the target heart rate; and
selecting, at the mobile device, a next audio selection from an audio library with a beats per minute similar to the beats per minute to adjust the heart rate.

45. The method of claim 44, wherein selecting the next audio selection from the audio library comprises:
identifying a potential next audio selection from the audio library;
analyzing the potential next audio selection to determine whether the beats per minute of the potential next audio selection substantially matches the beats per minute to adjust the heart rate; and
when the beats per minute of the potential next audio selection substantially matches the beats per minute to adjust the heart rate, selecting the potential next audio selection from the audio library as the next audio selection.

46. The method of claim 45, wherein analyzing the potential next audio selection comprises performing acoustic analysis of the potential next audio selection.

47. The method of claim 45, further comprising:
storing a history of audio selections played to the user; and
when the potential next selection is in the history of audio selections, selecting a different potential next selection to analyze beats per minute.

48. The method of claim 44, further comprising:
presenting a notification to the user when the difference between the current heart rate and the target heart rate is larger than the threshold value.

49. The method of claim 48, further comprising:
when a timeout period expires after the notification is presented to the user, evaluating the difference between the current heart rate and the target heart rate to determine whether it has been larger than the threshold value for the timeout period, and is so, performing the identifying the beats per minute of the current audio selection, determining the beats per minute to adjust the heart rate of the user from the current heart rate toward the target heart rate, and selecting the next audio selection from the audio library.

50. An apparatus to track heart rate for music selection comprising:
means for identifying, at a mobile device, a target heart rate of a user for an activity session;
means for receiving a current heart rate of the user from a sensor coupled to the mobile device; and
means for when a difference between the current heart rate and the target heart rate is larger than a threshold value for a duration longer than a threshold time:
means for identifying, at the mobile device, a beats per minute of a current audio selection;
means for determining, at the mobile device, a beats per minute to adjust the heart rate of the user from the current heart rate toward the target heart rate; and
means for selecting, at the mobile device, a next audio selection from an audio library with a beats per minute similar to the beats per minute to adjust the heart rate.
